(19) United States
(12) Patent Application Publication
(10) Pub. No.: US 2026/0183342 A1
(43) Pub. Date: Jul. 2, 2026

(54) ALLOGENIC IMPLANTS FOR TREATMENT OF INFLAMMATORY CONDITIONS

(71) Applicant: ROBLES BIOCEUTICS LLC, San Diego, CA (US)

(72) Inventors: Thomas Ichim, San Diego, CA (US); Maria Robles, San Diego, CA (US); Emma Lin, San Diego, CA (US); Maxim D. Wheatley, Los Angeles, CA (US); Joseph Ellis, San Diego, CA (US); Howard J. Leonhardt, Mission Viejo, CA (US)

(21) Appl. No.: 19/434,684

(22) Filed: Dec. 29, 2025

Related U.S. Application Data
(60) Provisional application No. 63/739,402, filed on Dec. 27, 2024.

(51) Int. Cl. A61K 35/52; A61K 35/32; A61K 35/50; A61K 38/19; A61K 38/21; A61P 37/06

(52) U.S. Cl. CPC A61K 35/52; A61K 35/32; A61K 35/50; A61K 38/193; A61K 38/217; A61P 37/06

(57) ABSTRACT

Disclosed herein are methods of reducing pathological inflammation and effects thereof through administration of tissues associated with regenerative and/or tolerogenic activity. In some embodiments, such tissues can include placental, ocular, testicular, and osseous tissues. Augmentation of therapeutic effects of said tissues may be achieved through contact with Injury Mimetics.

### INCORPORATION BY REFERENCE TO ANY PRIORITY APPLICATIONS

This application claims the benefit of U.S. Provisional Ser. No. 63/739,402, filed Dec. 27, 2024, which is hereby incorporated by reference in its entirety. Any and all applications for which a foreign or domestic priority claim is identified in the Application Data Sheet as filed with the present application are hereby incorporated by reference under 37 CFR 1.57.

### RELATED FIELD

Aspects of the present disclosure relate generally to the field of inflammation, as well as anti-inflammatory and regenerative treatments. In some embodiments, the disclosure related to methods and treatment for enhanced tissue healing. In some embodiments, the methods and/or treatment comprise a graft containing fragments of regenerative tissues.

### BACKGROUND

Mesenchymal stem cells possess anti-inflammatory properties, which is the basis for their efficacy in conditions such as graft versus host disease and other autoimmune conditions.

The physiopathology of septic shock is particularly complex and involves both pro-inflammatory and anti-inflammatory cytokines simultaneously, as well as immunoparalysis. There is no specific treatment for septic shock due to the inability of conventional pharmaceutical molecules to act on both the inflammatory aspect and on the anti-inflammatory aspect, while boosting the immune system.

### SUMMARY OF CERTAIN INVENTIVE ASPECTS

Aspects of the disclosure relate to implants with use for treating, preventing, or ameliorating a disease, disorder, or condition associated with inflammation. In some embodiments, the implant is allogenic. In some embodiments, the implant has use in treating an at least one inflammatory condition. In some embodiments, the implant comprises an at least one regenerative and/or tolerogenic tissue. In some embodiments, the tissue is pre-treated with an at least one injury mimetic. In some embodiments, the tissue is mammalian and/or human. In some embodiments, the at least one regenerative and/or tolerogenic tissue has use in treating an inflammatory disease, disorder, or condition.

Also disclosed herein is a composition with use against a disease, disorder, or condition associated with inflammation. In some embodiments, the composition comprises a therapeutic tissue, wherein the therapeutic tissue is pre-treated with an injury mimetic. In some embodiments, an injury mimetic comprises a molecule, hormone, factor, protein, cell composition, or any combination thereof, that is associated with an injury, produced following an injury, and/or is part of a subject's response to injury. In some embodiments, the injury mimetic comprises an interferon-gamma. In some embodiments, the composition comprises an interferon-gamma at a concentration and duration to increase expression of PD-L1 by more than about 25% in the endothelial cells within the therapeutic tissue. In some embodiments, the therapeutic tissue comprises a stem cell. In some embodiments, the tissue comprises at least one of: placental tissue, bone tissue, testis tissue, anterior chamber tissue, or any combination thereof. In some embodiments, the therapeutic tissue is chorionic and/or allogeneic. In some embodiments, the therapeutic tissue has regenerative activity and/or tolerogenic activity. In some embodiments, the injury mimetic comprises a TLR 2 toll-like receptor agonist. In some embodiments, the TLR 2 toll-like receptor agonist is beta glucan and/or a TLR 2 toll-like receptor. In some embodiments, the composition further comprises at least one of: a growth factor, signaling protein, senolytic agent, or any combination thereof. In some embodiments, the therapeutic tissue is pretreated with hyaluronic acid at about 5% to about 50% total concentration. In some embodiments, the composition further comprises G-CSF.

Also disclosed herein is a device for administering the composition of any one of the embodiments of the present disclosure to a subject in need thereof. In some embodiments, the device comprises a porous structure capable of facilitating growth of vascular and/or connective tissue. In some embodiments, the device further comprises an immunologically compatible mesh. “Immunologically compatible” is given its standard meaning, and can thus include a mesh that does not react with and/or activate a subject's immune response. In some embodiments, an immunologically compatible mesh is not recognized by the subject's immune system. In some embodiments, an immunologically compatible mesh reduces the subject's immune response.

Also disclosed herein is a method of treating, reducing, or preventing injury in a subject in need thereof. In some embodiments, the method comprises administering the composition of any one of the embodiments of the present disclosure, or the device of any one of the embodiments of the present disclosure, to the subject such that the composition and/or device is infiltrated with the subject's vascular and/or connective tissue. In some embodiments, the method further comprises co-administering at least one of: a growth factor, signaling protein, senolytic agent, or any combination thereof, to the subject. In some embodiments, the treatment results in reducing inflammation or a side effect from inflammation. In some embodiments, the administration comprises implantation. In some embodiments, the administration is performed subcutaneously.

Also disclosed herein is a method of accelerating healing in a subject in need thereof. In some embodiments, the method comprises administering the composition of any one of the embodiments of the present disclosure, or the device of any one of the embodiments of the present disclosure, to the subject such that the composition and/or device is infiltrated with the subject's vascular and/or connective tissue. In some embodiments, the method further comprises co-administering at least one of: a growth factor, signaling protein, senolytic agent, or any combination thereof, to the subject. In some embodiments, the treatment results in reducing inflammation or a side effect from inflammation. In some embodiments, the administration comprises implantation. In some embodiments, the administration is performed subcutaneously.

Also disclosed herein is a method of reducing inflammation in a subject in need thereof. In some embodiments, the method comprises administering the composition of any one of the embodiments of the present disclosure, or the device of any one of the embodiments of the present disclosure, to the subject such that the composition and/or device is infiltrated with the subject's vascular and/or connective tissue. In some embodiments, the method further comprises co-administering at least one of: a growth factor, signaling protein, senolytic agent, or any combination thereof, to the subject. In some embodiments, the treatment results in reducing inflammation or a side effect from inflammation. In some embodiments, the administration comprises implantation. In some embodiments, the administration is performed subcutaneously.

Also disclosed herein is a method of treating a disease, disorder, or condition associated with inflammation in a subject in need thereof. In some embodiments, the method comprises administering to the subject a tissue associated with regenerative and/or tolerogenic activity. In some embodiments, the treatment results in reducing inflammation or a side effect from inflammation. In some embodiments, the administration comprises implantation. In some embodiments, the administration is performed subcutaneously.

Also disclosed herein is a method of treating a disease, disorder, or condition associated with inflammation in a subject in need thereof. In some embodiments, the method comprises administering to the subject the composition of any one of the embodiments of the present disclosure, or the device of any one of the embodiments of the present disclosure. In some embodiments, the treatment results in reducing inflammation or a side effect from inflammation. In some embodiments, the administration comprises implantation. In some embodiments, the administration is performed subcutaneously.

Also disclosed herein is a method of reducing cytokine storm reactions by perfusing said tissue with interferon-gamma at a concentration and duration to increase expression of PD-L1 by more than about 25% in the endothelial cells within that tissue. In some embodiments, the interferon-gamma is an injury mimetic.

Also disclosed herein is a method of decreasing inflammation by administering amniotic membrane tissue while concurrently administering protein growth factors to mobilize endogenous stem cells. In some embodiments, said mobilization is elicited by administration of G-CSF.

Also disclosed herein is a treatment comprising co-administering to a subject in need thereof an implant according to any one of the embodiments of the present disclosure with an at least one growth factor, signaling protein, or senolytic agent. In some embodiments, the co-administration is performed on an at least one tissue in order to further reduce inflammation at the therapeutic site.

### BRIEF DESCRIPTION OF THE DRAWINGS

The features and advantages of the methods described herein will become apparent from the following description, taken in conjunction with the accompanying drawings. These drawings depict certain aspects of the methods described in the present application and, thus, are not to be considered limiting. In the drawings, similar reference numbers or symbols typically identify similar components, unless context dictates otherwise. The drawings may not be drawn to scale.

FIG. 1 illustrates a graph demonstrating the impact of control and treatment effects on mouse survival.

FIG. 2 illustrates a graph demonstrating the impact of control and treatment effects on mouse survival.

### DETAILED DESCRIPTION OF SOME EMBODIMENTS

Aspects of the present disclosure provide, among other things, a method of treating severe inflammation which historically has been resistant to therapy. Additionally, a method of administering allogeneic tissues and/or regenerative tissues for the amelioration of the pathological effects of inflammation. In some embodiments, the method comprises inclusion of specific growth factors to further promote healing. In some embodiments, the method comprises administration of senolytic agents to enhance recovery following inflammatory damage. In some embodiments, the method comprises signaling proteins to disrupt or reduce inflammation cause by cytokine storm reactions at the site of implantation.

Also disclosed herein are implants comprising an at least one allogeneic tissue and/or reconstituted tissue. In some embodiments, the tissue comprises placental tissue. In some embodiments, the placental tissue is chorionic placental tissue. In some embodiments, the tissue is capable of producing an at least one anti-inflammatory mediator. In some embodiments, the tissue is treated with an injury mimetic prior to being administered to a subject. In some embodiments, the treatment is ex vivo or in vivo. In some embodiments, the injury mimetic comprises a tlr agonist. In some embodiments, the tissue comprises at least 1, 2, or 3 of: an allogeneic bone, testis, and an anterior chamber tissue. In some embodiments, the tissue is pre-treated with an at least one hormone and/or growth factor. In some embodiments, the implant is co-administered to a subject with an at least one hormone and/or growth factor. In some embodiments, the growth factor comprises GM-CSF. In some embodiments, the implant is co-administered to a subject with an at least one senolytic agent. In some embodiments, the senolytic agent has use in enhancing recovery of a subject from post-inflammatory damage.

Aspects of the disclosure relate to the use of an implanted, porous scaffold device to deliver the therapeutic effects of the tissue. In some embodiments, such as device contains the tissue and other relevant agents in order to effectively elicit the desired therapeutic effects.

### Nonlimiting Example Therapeutic Tissue

Aspects of the present disclosure relate to a therapeutic tissue with use in treating, preventing, or reducing the symptoms associated with an inflammatory disease, disorder, or condition. In some embodiments, the therapeutic tissue has use in reducing inflammation and/or an at least one side effect from inflammation. In some embodiments, the therapeutic tissue comprises at least one of: chorionic placental tissue, allogeneic bone tissue, testis tissue, anterior chamber tissue, or tissue from regenerative or tolerogenic tissue. In some embodiments, the therapeutic tissue comprises placenta tissue. In some embodiments, the therapeutic tissue comprises a stem cell. In some embodiments, the stem cell is a mesenchymal stem cell.

Mesenchymal stem cells (MSCs) have anti-inflammatory properties which can reduce pain and swelling around sites of injury or infection. Some tissue types contain higher concentrations of stem cells and present as a target for reducing inflammation at an injury site. Numerous studies using mouse models of endotoxaemia and peritonitis have revealed the ability of MSCs to reduce plasma levels of IL6, IL1β, IL12, IL2 and IL 17 and to lower the tissular concentration of TNFα, IL6, IL1β, IL12 in the lungs, liver and intestine or in the bronchoalveolar fluid.

The pathophysiology of inflammation is complex and involves competing pro-inflammatory and anti-inflammatory responses that are driven by a number of distinct mechanisms. In some embodiments, additional substances, such as growth factors, signaling proteins, and senolytic agents are administered concurrently with the regenerative tissue to further downregulate the inflammatory response.

In some embodiments, the “priming” of therapeutic tissue, but not single cells, with Injury Mimetics induces a potent anti-inflammatory reflex which is uniquely capable of blocking sepsis and severe inflammation. In some embodiments, the three dimensional tissue structure, as well as peripheral non-stem cell cells, act as adjuvants of anti-inflammatory activity in a subject in need thereof.

### Terms

In the present disclosure, reference is made to the accompanying drawings, which form a part hereof. In the drawings, similar symbols typically identify similar components, unless context dictates otherwise. The illustrative embodiments described in this disclosure, including the drawings and claims, are not meant to be limiting. Some embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

The section headings used herein are for organizational purposes only and are not to be construed as limiting the subject matter described.

The articles “a” and “an” are used herein to refer to one or to more than one (for example, at least one) of the grammatical object of the article, unless the context dictates otherwise. By way of example, “an element” means one element or more than one element.

By “about” is meant a quantity, level, value, number, frequency, percentage, dimension, size, amount, weight or length that is approximately the recited value. Where it is not clear from the context what is encompassed by “about,” it will mean the value recited+/−10%.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it includes, but is not limited to, primates, including simians (chimpanzees, apes, monkeys) and humans, cattle, horses, sheep, goats, swine, rabbits, dogs, cats, rodents, rats, mice, guinea pigs, or the like.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” has its plain and ordinary meaning as understood in light of the specification and refers to the performance of a method inside living organisms, usually animals, mammals, including humans, and plants, as opposed to a tissue extract or dead organism.

As used herein, “ex vivo” has its plain and ordinary meaning as understood in light of the specification and refers to the performance of a method outside a living organism with little alteration of natural conditions.

As used herein, “in vitro” has its plain and ordinary meaning as understood in light of the specification and refers to the performance of a method outside of biological conditions, e.g., in a petri dish or test tube.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the terms “treating” or “treatment” have their plain and ordinary meaning as understood in light of the specification, and refer to an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (e.g., not worsening) the state of disease, prevention of a disease's transmission or spread, delaying or slowing of disease progression, amelioration or palliation of the disease state, diminishment of the recurrence of disease, and remission, whether partial or total and whether detectable or undetectable. “Treating” and “treatment” as used herein also include prophylactic treatment. Treatment methods include administering to a subject a therapeutically effective amount of an active agent. The administering step may include a single administration or may include a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

The term “administering” includes oral administration, topical contact, administration as a suppository, intravenous, intraperitoneal, intramuscular, intralesional, intra-tumoral, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intra-tumoral, intraventricular, and intracranial. Other modes of delivery include, but are not limited to, the use of liposomal formulations, intravenous infusion, transdermal patches, etc. By “co-administer” it is meant that a first compound described herein is administered at the same time, just prior to, or just after the administration of a second compound described herein.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or carrier can refer to a diluent, adjuvant, excipient, or vehicle with which the pharmaceutical formulation is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include sugars, starch, glucose, fructose, lactose, sucrose, maltose, gelatin, malt, rice, flour, chalk, silica gel, sodium stearate, glycerol monostearate, talc, salts, sodium chloride, dried skim milk, glycerol, propylene, glycol, water, ethanol and the like. A non-limiting example of a physiologically acceptable carrier is an aqueous pH buffered solution. The physiologically acceptable carrier may also include one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as glycerol, erythritol, threitol, arabitol, xylitol, ribitol, mannitol, sorbitol, galactitol, fucitol, iditol, inositol, isomalt, maltitol, or lactitol, salt-forming counterions such as sodium, and nonionic surfactants such as TWEEN®, polyethylene glycol (PEG), and PLURONICS®. The formulation, if desired, can also contain minor amounts of wetting, bulking, emulsifying agents, or pH buffering agents. These formulations can take the form of solutions, suspensions, emulsion, sustained release formulations and the like. The formulation should suit the mode of administration.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

The terms “first,” “second,” and “third” used in combination with substances are intended to designate distinguishable features to similar substances and do not imply any particular order unless otherwise specified.

The term “toll-like receptor” as used herein refers without limitation to a broad class of receptors which influence immunoinflammatory responses.

The term “toll-like receptor agonist” as used herein refers without limitation to a chemical or class of chemicals that activate toll-like receptors similarly to endogenous signaling molecules.

The term “beta-glucan” as used herein refers to a class of glucose polymers in the beta-isomer configuration. For example, without limitation, β-1,3-glucan is a common beta-glucan of interest in immunomodulatory molecule of interest.

The term “cytokine” as used herein refers without limitation to a broad class of proteins that facilitates intercellular signaling, particularly, but without limitation, in immunoinflammatory signaling.

The term “cytokine storm” as used herein refers without limitation to a category of symptoms associated with immune dysregulation of cytokine signaling, including systemic inflammation, multiorgan dysfunction or failure, or other constitutional symptoms.

The term “growth factor” as used herein is a broad term and is to be given its ordinary and customary meaning to a person of ordinary skill in the art (and is not to be limited to a special or customized meaning), and refers without limitation to a class of proteins or other molecules which stimulate cellular growth and which can induce cell proliferation and differentiation, tissue growth and repair, and healing.

The term “senolytic” as used herein is a broad term and is to be given its ordinary and customary meaning to a person of ordinary skill in the art (and is not to be limited to a special or customized meaning), and refers without limitation to a class of therapies which help clear senescent cells.

The term “graft” as used herein is to be given its ordinary and customary meaning to a person of ordinary skill in the art (and is not to be limited to a special or customized meaning), and refers without limitation to healthy tissue which is used to replace or treat diseased, injured, or otherwise damage tissue.

The term “scaffold” as used herein is to be given its ordinary meaning to a person of ordinary skill in the art (and is not to be limited to a special or customized meaning), and refers without limitation to porous materials or structures which is suitable for structural support and/or providing an environment conducive to cell activity, such as a material or structure which mimics an extracellular matrix in biological tissue or organ systems.

The term “therapeutic effect” as used herein is to be given its ordinary meaning to a person of ordinary skill in the art (and is not to be limited to a special or customized meaning) and refers without limitation to beneficial or positive outcomes resulting from a therapy or treatment.

As described herein, this disclosure teaches a method of administering three dimensional tissue fragments of regenerative tissue comprising a graft to produce a marked therapeutic effect against inflammatory conditions. This disclosure teaches that administration of various “injury mimetics,” senolytics, or other agents to said tissue prior to implantation augments anti-inflammatory and regenerative activity. This disclosure teaches the use of a device to effectively apply the tissue and produce the desired therapeutic effects against inflammation at the site of implantation.

In some embodiments, the present disclosure is directed to a tissue graft possessing anti-inflammatory activity wherein said graft comprises dehydrated tissue having a top and bottom surface and an outer contour sized and shaped for use in a suitable medical procedure, wherein a texture or design is embossed within the dehydrated tissue and wherein the embossment distinguishes the top from the bottom surface of the tissue; and wherein the dehydrated tissue graft is usable in the suitable medical procedure after being rehydrated. In some embodiments, the dehydrated tissue comprises either an amniotic membrane layer or a chorion tissue layer. In some embodiments, the dehydrated tissue comprises two or more layers of amniotic membrane and chorion tissue, wherein the two or more layers include a plurality of amniotic membrane, a plurality of chorion tissue, or a plurality of amniotic membrane and chorion tissue.

In some embodiments, the disclosed graft is comprised of regenerative tissue, such as placental tissue. For example, in some embodiments chorionic placental tissue is used. In some embodiments, the graft is comprised of different types of regenerative tissue, including but not limited to, allogeneic bone tissue, testis tissue, and/or anterior chamber tissue is used.

In some embodiments, the graft is administered subcutaneously. In some embodiments, the tissue comprising the graft is pre-treated with a senolytic agent prior to implantation. For example, in some embodiments, hyaluronic acid at a 1-70% concentration prior to implantation. For example, in some embodiments the concentration of hyaluronic acid is 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70% or a range defined by any two numbers therein. In some embodiments, the tissue comprising the graft is pre-treated with a toll-like receptor agonist to further decrease inflammation at the site of implantation. In other embodiments, the graft is pre-treated with different toll-like receptor agonists, including but not limited to toll-like receptors belonging to the TLR 1, TLR 2, TLR 3, TLR 4, TLR 5, TLR 6, TLR 7, TLR 8, TLR 9, TLR 10, TLR 11, TLR 12, or TLR 13. For example, the TLR 2 toll-like receptor beta-glucan was used to treat the graft in an example embodiment of the disclosure.

In some embodiments, the tissue comprising the graft is perfused with immunoregulatory molecules to suppress cytokine storm reactions. For example, in some embodiments, a graft can be perfused with anti-inflammatory cytokines, including but not limited to interferon-gamma, to reduce the resulting cytokine storm reaction at the site of implantation. In some embodiments, the graft is perfused with interform-gamma at a concentration and duration to increase the expression of PD-L1 by at least 25% within the endothelial cells found within the tissue which comprises the graft. In some embodiments, the graft is perfused with interferon-gamma at a concentrate to elicit an increase in PD-L1 by 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% or more, or a range defined by any two numbers therein or thereafter.

In some embodiments, the tissue is placed in a device which comprises: a porous scaffold comprising an immunologically compatible polymer mesh forming the walls of at least one chamber, wherein the chamber comprises an opening at either or both of a proximal end and a distal end of the chamber, wherein the proximal end and the distal end are separated by a lumen that is bounded by the walls, and wherein the porous scaffold has pores sized to facilitate growth of vascular and connective tissues around and through the walls of the at least one chamber; at least one removable, non-porous plug configured to be positioned within the lumen of the at least one chamber, wherein the plug extends along the lumen of the chamber; and at least one seal configured to enclose either or both the proximal end and the distal end of the chamber; maintaining the device in the patient's body until the device is infiltrated with vascular and connective tissues; accessing the implanted device; withdrawing the plug; and infusing the chamber with cells from the regenerative tissue.

In some embodiments, tissue is introduced while concurrently administering a growth factor to mobilize endogenous stem cells in order to further decrease inflammation. Growth factors can come from a variety of sources, including from the platelet-derived growth factor family, vascular endothelial growth factor family, epidermal growth factor family, fibroblast growth factor family, hepatocyte growth factor family, neurotrophin family, ephrin family, agrin family, GDNF family, insulin family are administered concurrently to regenerative tissue in order to further reduced inflammation at the site of implantation. For example, in some embodiments tissue is administered in conjunction with a colony stimulating factor (CSF), including but limited to G-CSFs, GM-CSFs, and M-CSFs.

Aspects of the present disclosure can also be found by way of the following enumerated embodiments:
1. A composition with use against a disease, disorder, or condition associated with inflammation, the composition comprising a therapeutic tissue, wherein the therapeutic tissue is pre-treated with an injury mimetic.
2. The composition of embodiment 1, wherein the therapeutic tissue comprises a stem cell.
3. The composition of embodiment 1 or 2, wherein the tissue comprises at least one of: placental tissue, bone tissue, testis tissue, anterior chamber tissue, or any combination thereof.
4. The composition of any one of the preceding embodiments, wherein the therapeutic tissue is chorionic and/or allogeneic.
5. The composition of any one of the preceding embodiments, wherein the therapeutic tissue has regenerative activity and/or tolerogenic activity.
6. The composition of any one of the preceding embodiments, wherein the injury mimetic comprises a TLR 2 toll-like receptor agonist.
7. The composition of embodiment 6, wherein the TLR 2 toll-like receptor agonist is beta glucan and/or a TLR 2 toll-like receptor.
8. The composition of any one of the preceding embodiments, further comprising at least one of: a growth factor, signaling protein, senolytic agent, or any combination thereof.
9. The composition of any one of the preceding embodiments, wherein the therapeutic tissue is pretreated with hyaluronic acid at about 5% to about 50% total concentration.
10. A device for administering the composition of any one of embodiments 1-9 to a subject in need thereof, the device comprising a porous structure capable of facilitating growth of vascular and/or connective tissue.
11. The device of embodiment 10, further comprising immunologically compatible mesh.
12. A method of reducing inflammation in a subject in need thereof, the method comprising administering the composition of any one of embodiments 1-9, or the device of any one of embodiments 10-11, to the subject such that the composition and/or device is infiltrated with the subject's vascular and/or connective tissue.
13. The method of embodiment 12, further comprising of co-administering at least one of: a growth factor, signaling protein, senolytic agent, or any combination thereof, to the subject.
14. A method of treating a disease, disorder, or condition associated with inflammation in a subject in need thereof, comprising:
   administering to the subject a tissue associated with regenerative and/or tolerogenic activity.
15. A method of treating a disease, disorder, or condition associated with inflammation in a subject in need thereof, comprising:
   administering to the subject the composition of any one of embodiments 1-9, or the device of any one of embodiments 10-11.
16. The method of embodiment 14-15, wherein the treatment results in reducing inflammation or a side effect from inflammation.
17. The method of any one of embodiments 12-16, wherein the administration comprises implantation.
18. The method of any one of embodiments 12-17, wherein the administration is performed subcutaneously.
19. A method of reducing cytokine storm reactions by perfusing said tissue with interferon-gamma at a concentration and duration to increase expression of PD-L1 by more than about 25% in the endothelial cells within that tissue.
20. A method of decreasing inflammation by administering amniotic membrane tissue while concurrently administering protein growth factors to mobilize endogenous stem cells.
21. The method of embodiment 20, wherein said mobilization is elicited by administration of G-CSF.

### EXAMPLES

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends, and advantages inherent herein. Changes therein and other uses which are encompassed within the spirit of the disclosure as defined by the scope of the claims will occur to those skilled in the art.

### Example 1

A 48-year-old male patient with myeloid leukemia who had previously had a stem cell transplant presented with apparent septicemia. On day 5 post-presentation he had a fever (40° C.). His blood culture showed the presence of alpha streptococci. His hemoglobin had decreased from 105 g/l to 63 g/l. The patient was dependent on ventilatory support. 10 grams of chorionic placental tissue which had been pretreated with 1000 IU of interferon gamma for 1 hour was dissected in 1 mm (2) pieces was implanted in his abdominal area. Following treatment, the patient's oxygen saturation increased from 87-91% to 97% (and was thereby stabilized) approximately 8 hours after implantation. Chest radiography improved and was normal on day 21 after transplant.

### Example 2

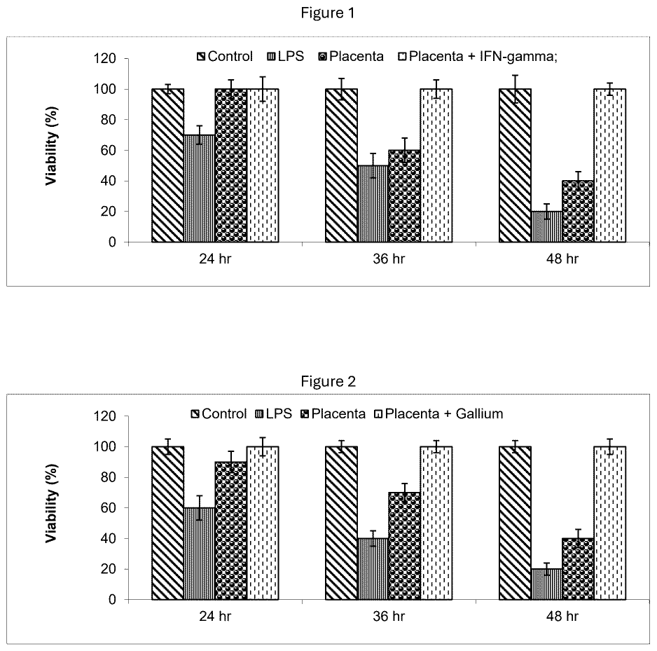

Female BALB/c mice of 10-15 weeks of age where administered saline (Control), lipopolysaccharide (100 micrograms per mouse) (LPS), LPS and 2 mm (3) human chorionic placental tissue subcutaneously (placenta), and LPS plus 2 mm (3) human chorionic placental tissue pretreated with interferon gamma 100 ng/ml for 1 hour. Viability was assessed as seen in FIG. 1.

### Example 3

Female BALB/c mice of 10-15 weeks of age where administered saline (Control), lipopolysaccharide (100 micrograms per mouse) (LPS), LPS and 2 mm (3) human chorionic placental tissue subcutaneously (placenta), and LPS plus 2 mm (3) human chorionic placental tissue pretreated with gallium maltolate 20 ng/ml for 1 hour. Viability was assessed as seen in FIG. 2.

### Example 4

Chorionic tissue was dissected, minced into 1 mm³ chunks under sterile conditions, and washed thrice in phosphate-buffered saline (PBS) to remove debris. Chunks were incubated in culture medium (DMEM with 10% FBS) supplemented with 5 micrograms/mL recombinant HMGB1 for 48 hours at 37° C. in 5% CO₂.

Non-treated chunks underwent identical washing and incubation without IFN-γ.

Experimental autoimmune encephalomyelitis was induced in 8-10-week-old female C57BL/6 mice (n=30, 10 per group) via subcutaneous immunization with 200 μg MOG₃₅₋₅₅ peptide emulsified in complete Freund's adjuvant containing 4 mg/mL heat-killed Mycobacterium tuberculosis, followed by 200 ng pertussis toxin intraperitoneally on days 0 and 2. On day 7 post-induction (pre-clinical onset), 2 chunks (total ˜2 mm³) per mouse were implanted subcutaneously into the flank under anesthesia (ketamine/xylazine).

Mice were divided into 3 groups: (1) Control (placebo: PBS injection); (2) Non-IFN-treated placenta chunks; (3) IFN-γ-treated placenta chunks.

Clinical scoring occurred daily from day 0 to 30 using a standard 0-5 scale: 0=no signs; 1=limp tail; 2=hindlimb weakness; 3=hindlimb paralysis; 4=forelimb involvement/quadriplegia; 5=moribund/death.

As shown in the below Table 1, TLR4-activated placental chunks were capable of reducing autoimmunity:

TABLE 1

| | | | | | | | | | | | Average ± |
|---|---|---|---|---|---|---|---|---|---|---|---|
| Group | Mouse 1 | Mouse 2 | Mouse 3 | Mouse 4 | Mouse 5 | Mouse 6 | Mouse 7 | Mouse 8 | Mouse 9 | Mouse 10 | SEM |
| Control | 3.0 | 4.0 | 3.5 | 4.0 | 5.0 | 3.0 | 4.5 | 3.5 | 4.0 | 3.0 | 3.75 ± |
| (Placebo) | | | | | | | | | | | 0.21 |
| Non- | 1.5 | 2.0 | 1.0 | 2.5 | 1.5 | 2.0 | 1.0 | 2.0 | 1.5 | 2.0 | 1.70 ± |
| HMGB1 | | | | | | | | | | | 0.16 |
| Treated | | | | | | | | | | | |
| Placenta | | | | | | | | | | | |
| HMGB1 | 0.0 | 0.5 | 0.0 | 0.0 | 0.5 | 0.0 | 0.0 | 0.0 | 0.5 | 0.0 | 0.15 ± |
| Treated | | | | | | | | | | | 0.06 |
| Placenta | | | | | | | | | | | |

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 markers refers to groups having 1, 2, or 3 markers. Similarly, a group having 1-5 markers refers to groups having 1, 2, 3, 4, or 5 markers, and so forth.

All patent filings, websites, other publications, accession numbers and the like cited above or below are incorporated by reference in their entirety for all purposes to the same extent as if each individual item were specifically and individually indicated to be so incorporated by reference. Any feature, step, element, embodiment, or aspect disclosed herein can be used in combination with any other unless specifically indicated otherwise.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a formulation having at least one of A, B, and C” would include but not be limited to formulations that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a formulation having at least one of A, B, or C” would include but not be limited to formulations that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. For example, “about 5”, shall include the number 5. Finally, as will be understood by one skilled in the art, a range includes each individual member. Thus, for example, a group having 1-3 values refers to groups having 1, 2, or 3 values. Similarly, a group having 1-5 values refers to groups having 1, 2, 3, 4, or 5 values, and so forth.

It would be understood that the various sizes, materials, configurations and arrangements disclosed herein may be combined and constructed in any way that is feasible to create a hybrid for any particular end use. Accordingly, all suitable modifications and equivalents may be resorted to falling within the scope of the appended claims. Unless defined otherwise, all technical and scientific terms used herein have same meaning as commonly understood by one of ordinary skill in the art to which this application belongs. Also, as used herein and in the appended claims, the singular form “a”, “and”, and “the” include plural referents unless the context clearly dictates otherwise.

It is to be understood that the present invention is not to be limited to the exact description and embodiments as illustrated and described herein. To those of ordinary skill in the art, one or more variations and modifications will be understood to be contemplated from the present disclosure. Accordingly, all expedient modifications readily attainable by one of ordinary skill in the art from the disclosure set forth herein, or by routine experimentation therefrom, are deemed to be within the true spirit and scope of the invention as defined by the appended claims.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A composition with use against a disease, disorder, or condition associated with inflammation, the composition comprising a therapeutic tissue, wherein the therapeutic tissue is pre-treated with an injury mimetic.

2. The composition of claim 1, wherein the therapeutic tissue comprises a stem cell.

3. The composition of claim 1, wherein the tissue comprises at least one of: amniotic membrane tissue, placental tissue, bone tissue, testis tissue, anterior chamber tissue, or any combination thereof.

4. The composition of claim 1, wherein the therapeutic tissue is chorionic, allogeneic, or both.

5. The composition of claim 1, wherein the therapeutic tissue has regenerative activity, tolerogenic activity, or both.

6. The composition of claim 1, wherein the injury mimetic comprises a TLR 2 toll-like receptor agonist.

7. The composition of claim 6, wherein the TLR 2 toll-like receptor agonist comprises a beta glucan, a TLR 2 toll-like receptor, or both.

8. The composition of claim 1, further comprising at least one of: a growth factor, signaling protein, senolytic agent, or any combination thereof.

9. The composition of claim 1, wherein the therapeutic tissue is pretreated with hyaluronic acid at about 5%, about 50%, or any value that is between about 5% and about 50%, total concentration.

10. A device for administering a composition to a subject in need thereof, the device comprising a porous structure capable of facilitating growth of vascular and/or connective tissue, wherein the composition comprises a therapeutic tissue that is pre-treated with an injury mimetic.

11. The device of claim 10, further comprising an immunologically compatible mesh.

12. A method of treating a disease, disorder, or condition associated with inflammation in a subject in need thereof, the method comprising administering to the subject a composition, such that the composition is infiltrated with the subject's vascular and/or connective tissue, wherein the composition comprises a therapeutic tissue associated with regenerative activity, tolerogenic activity, or both.

13. The method of claim 12, wherein the therapeutic tissue is pre-treated with an at least one injury mimetic.

14. The method of claim 12, further comprising of co-administering at least one of:
a growth factor, signaling protein, senolytic agent, or any combination thereof, to the subject.

15. The method of claim 12, wherein the treatment results in reducing inflammation or a side effect from inflammation.

16. The method of claim 12, wherein the administration comprises implantation.

17. The method of claim 12, wherein the administration is performed subcutaneously.

18. The method of claim 12, wherein the treatment results in reducing, preventing, or eliminating a cytokine storm reaction in the subject.

19. The method of claim 12, wherein the composition comprises interferon-gamma at a concentration and duration to increase expression of PD-L1 by at least about 25% in an endothelial cell.

20. The method of claim 12, wherein the composition comprises a protein growth factor with use in mobilizing endogenous stem cells, optionally wherein the protein growth factor is G-CSF.
